(19) United States
(12) Patent Application Publication
(10) Pub. No.: US 2026/0256771 A1
(43) Pub. Date: Sep. 3, 2026

(54) GALUNISERTIB FOR USE IN THE TREATMENT OF ANDROGENIC ALOPECIA

(71) Applicant: SAÚDE VIÁVEL, S.A., PORTO (PT)

(72) Inventors: Elsa Clara CARVALHO LOGARINHO SANTOS, PORTO (PT); Ana Rita DE SOUSA CASTRO, PORTO (PT)

(21) Appl. No.: 18/875,374

(22) PCT Filed: Jun. 19, 2023
(86) PCT No.: PCT/PT2023/050015

§ 371 Date: Dec. 16, 2024

(30) Foreign Application Priority Data
Jun. 17, 2022 (PT) 118057
Jun. 12, 2023 (PT) 118716

(51) Int. Cl. A61K 31/4709; A61P 17/14

(52) U.S. Cl. CPC A61K 31/4709; A61P 17/14

(57) ABSTRACT

A potent inhibitor of the transforming growth factor beta (TGFβ) receptor I (TβRI), galunisertib (LY2157299), for use in the treatment of androgenic alopecia (AGA), acting against TGFβ repressive signaling during ex vivo human HF growth cycle. Inhibition by galunisertib translates into improved proliferation and β-catenin levels in the hair bulb epithelial compartment and delayed catagen onset. Moreover, topical treatment with galunisertib in a depilated mouse model, prolongs the hair cycle anagen stage, enhancing hair growth without apparent side effects. Histological studies further confirm that galunisertib treatment improves the proliferation of hair germ progenitor cells. The use, compositions and routes of administration may be advantageously employed for example as a topical composition for pharmacological intervention for use in the treatment of AGA, or any other types of alopecia or hair loss.

### TECHNICAL FIELD OF THE INVENTION

The present invention relates to the technical field of human health; medical science; preparations for medical purposes; in particular, medicinal preparations containing organic active ingredients, namely heterocycles. Specifically, this invention relates to a new treatment for hair loss.

### PRIOR ART

Typically, hair follicle (HF) growth and regeneration involve a huge set of hormones and growth factors that act synergically on stem cell niches of the dermal compartment, the dermal papilla (DP) and the dermal sheath (DS). DP is at the basis of the HF and is surrounded by epithelial matrix cells. DP cells (DPCs) provide instructive signals required to activate epithelial progenitors and regulate the HF cycle, which consists in phases of growth (anagen), apoptotic-driven regression (catagen), and rest (telogen) (Cotsarelis, 2006). Importantly, hair loss (also known as alopecia or baldness) is the phenotypic outcome of dysfunctional dermal trichogenic signaling caused by depletion of DPCs. The balding process is associated with shorter anagen, longer telogen phases, as well as with a gradual reduction in the HF size that directly correlates with DPC number (Pantelireis & Higgins, 2018).

Androgenic alopecia (AGA) is characterized by HF miniaturization (reduction in diameter, length and pigmentation) during repeated hair cycles with shortened anagen (Otberg, Finner, & Shapiro, 2007). Androgens, in particular dihydrotestosterone (DHT) generated by α5-reductase metabolization of testosterone, act via the DP in inducing hair loss (Martinez-Jacobo, Villarreal-Villarreal, Ortiz-López, Ocampo-Candiani, & Rojas-Martinez, 2018). The higher levels of type II α5-reductase and of androgen receptors (AR) in DPCs from balding vs. non-balding scalp HFs, drive a gene expression program that negatively impacts hair growth (Itami, Sonoda, Kurata, & Takayasu, 1994; Winiarska et al., 2006a). The DHT-AR complex alters the expression of key hair regulatory pathways, for example inhibiting the Wnt/β-catenin signaling pathway. Up to date, pharmacological treatments to AGA are still limited to the US Food and Drug Administration (FDA) approved Minoxidil (potassium channel agonist) and Finasteride (α5-reductase inhibitor) (Adil & Godwin, 2017). Although these drugs prove effective for some patients, they entail significant side effects such as skin itching and erectile dysfunction (Madaan, Verma, Singh, & Jaggi, 2018; Talavera-Adame, Newman, & Newman, 2017). Another mainstream treatment, hair transplant, is limited by the number of hair grafts in the donor region. Consequently, there is a high demand for more effective therapeutic solutions targeting AGA, ideally improving DP fitness.

### SUMMARY OF THE INVENTION

In the present invention we disclose that galunisertib, a specific TGFβ receptor type I tyrosine kinase inhibitor, inhibits TGFβ signaling in the DP, translating into improved cell proliferation and β-catenin expression in the hair bulb epithelial compartment and notably, delaying catagen onset. As such, the present invention discloses galunisertib for use in a pharmacological composition for the treatment of diseases selected from the list consisting of androgenic alopecia and or other types of hair loss, according to claim 1.

In another embodiment of the present invention, galunisertib for use in a pharmacological composition for use in the treatment of androgenic alopecia or other types of hair loss, is characterized by comprising any of the chemical derivatives of the general formula:

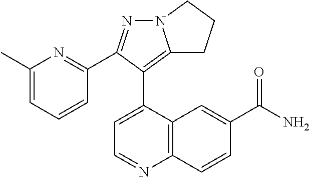

according to claim 2.

Another aspect of the present invention refers to a pharmaceutical composition characterized by comprising galunisertinib and other compounds selected from the group consisting of an excipient, a diluent, a stabilizing agent, a carrier, a surfactant, an antioxidant and a buffering agent, and combinations thereof, according to claim 3.

Another aspect of the present invention refers to a route of administration of the said composition in the said use which is characterized by, being selected from the group consisting of topical, dermal, transdermal and subcutaneous, according to claim 4.

### DETAILED DESCRIPTION OF THE INVENTION

The present invention embodies the therapeutic potential of Galunisertib, which can be obtained from commercial sources, such as Sigma Aldrich company, against human hair loss by counteracting TGFβ signaling activation from the DP to the epithelial compartment, thereby improving Wnt/β-catenin and Lef1 trichogenic signaling and delaying catagen in human HF organ cultures—HFOCs, as well as in depilated mice under topical treatment, supporting the use of this compound, or any of its derivatives, in a treatment for AGA or any other types of hair loss.

The present invention discloses that TGFβ signaling is activated in the DP of human HFs collected from non-balding scalp regions and cultured ex vivo. Despite being supposedly non-responsive to DHT, a comparative analysis is done between HFOCs untreated and treated with DHT (FIG. 1a). TGFβ signaling is composed of TGFβ1, TGFβ2, and TGFβ3 that, when bound to TGFβ type II receptor (TβRII), phosphorylate and activate the TGFβ type I receptor (TBRI/ALK-5) (Vander Ark, Cao, & Li, 2018). qRT-PCR quantitative analysis of TGFβ1, TGFβ2, TGFβ3 transcript levels in DP isolated from human HFs cultured ex vivo for 48 h reveals that only TGFβ2 is slightly increased in DHT-treated vs. untreated HFOCs, with TGFβ1 and TGFβ3 levels remaining unchanged (FIG. 1b). Activated TBRI phosphorylates SMAD2 and SMAD3, which then bind to SMAD4. The phospho-SMAD2/3-SMAD4 complex translocates into the nucleus to regulate canonical gene transcription (Vander Ark et al., 2018). To determine if TGFβ signaling pathway is indeed activated in HFOCs with and without DHT, the protein levels of phosphorylated SMAD2 (p-SMAD2) are measured in the hair bulbs. It's found that TGFβ signaling appears activated in both conditions, as inferred by the detection of p-SMAD2 (FIG. 1c). p-SMAD2 levels are equivalent in untreated and DHT-treated HFOCs, confirming that TGFβ signaling activation in human non-balding HF organ cultures occurs independently of DHT-AR triggering.

The present invention further discloses that Galunisertib restrains TGFβ signaling activation in HF organ cultures, characterizing the potential therapeutic role of TGFβ signaling on HF regression.

Galunisertib, which can be obtained from commercial sources, such as Eli Lilly company, can counteract TGFβ signaling and its possible detrimental effect on DP fitness and HF growth cycle, ultimately allowing its use for the treatment of AGA. In other embodiments, other chemical derivatives from the said chemical formula constitute, may be used in a treatment with the same purpose.

Titration of the optimal concentration of this compound using as readout the mean time (days) that HFOCs take to enter catagen shows that a working dose of 10 μM reveals capable of delaying catagen entry, in comparison to untreated controls, with an impact in the hair shaft elongation (FIG. 2a-c). A potential synergic effect of Galunisertib with minoxidil is excluded (FIG. 3a-c). Combination treatment does not potentiate the effect of each drug alone in catagen delay.

The present invention further discloses that Galunisertib decreases TGFβ2 in the bulb epithelial compartment of HFOCs. Although DHT induces TGFβ2 transcription in the DP, TGFβ2 protein has been suggested to paracrinally act on the adjacent epithelial compartment. Thus, to directly visualize TGFβ2 protein localization pattern in HFOCs with or without Galunisertib for 48 h, immunohistochemistry analysis is done in cryo-sectioned HFs (FIG. 4A). There isn't any significant change in the DP levels of TGFβ2 upon Galunisertib treatment (FIG. 4a,b). Also, the p-SMAD2 canonical TGFβ target is not significantly reduced (FIG. 4a,c). However, noticeably TGFβ2 protein is predominantly located in the adjacent epithelial hair matrix, pre-cortex and inner root sheath of human scalp HFs (FIG. 4a). Quantitative analysis of TGFβ2 and p-SMAD2 fluorescence intensity levels at the bulb epithelial compartment surrounding the DP reveals decreased levels in Galunisertib-treated HFOCs. This supports that induced TGFβ2 transcription in the DP of HFOCs is followed by TGFβ2 protein accumulation on the adjacent epithelial compartment. Galunisertib is found to inhibit this paracrine signaling.

The present invention also discloses that Galunisertib improves cell proliferation and β-catenin expression in the bulb epithelial compartment of HFOCs. Immunohistochemistry analysis in cryosections of HFOCs treated with Galunisertib for 48 h, reveals an increased number of bulb epithelial cells staining positive for the Ki67 proliferation marker, as well as higher Ki67 fluorescence intensity levels, in comparison to untreated HFOCs (FIG. 5a,b). Fluorescence intensity levels of the apoptosis marker cleaved Caspase 3, mainly detected in the DP, do not show significant changes, suggesting that Galunisertib acts by stimulating cell proliferation instead of inhibiting apoptosis (FIG. 5a,c). Moreover, Galunisertib significantly enhances β-catenin intensity levels in the DP adjacent epithelial compartment, suggesting for Wnt/β-catenin signaling activation (FIG. 6a,c,e). Notably, the transcription factor LEF1, a marker of DP cell trichogenicity, although not significantly upregulated in the DP, displays increased levels in the bulb epithelium upon Galunisertib treatment (FIG. 6a,b,d). Noteworthy, LEF1 is observed to also localize at the epidermal basement membrane surrounding the DP, which is a distinctive feature of human HF (FIG. 6a).

The present invention also discloses that TGFβ inhibition enhances hair growth in mice in vivo as assessed in 7-week-old C57BL/6 depilated male mice subjected to topical treatment with galunisertib at two different concentrations (10 and 100 μM), along with negative and positive controls: vehicle and 3% minoxidil topical treatments, respectively (FIG. 7a). Upon depilation-induced anagen (for hair cycle synchronization; day −2), hair growth starts within 7 days (day 5 post-treatment). Macroscopic assessment of hair growth indicates the depilated area to be consistently darker in galunisertib- and minoxidil-treated mice vs. control mice from day 5 to day 14, which supports an increased hair density (FIG. 7b). At day 9, magnified photographs of the depilated areas show marked improvement of hair growth in mice treated with 10 μM galunisertib, followed by the 100 μM galunisertib and 3% minoxidil treatments, in comparison to vehicle control (FIG. 7c). Noteworthy, no apparent adverse side effects are observed in any mice treated with galunisertib. Trichoscopy examination of the depilated regions at days 8 and 10 confirms the increased hair density in mice treated with galunisertib, in particular for the 10 μM concentration, supporting that this lower dose is sufficient to inhibit TGFβ signaling and optimal to impact the hair growth cycle (FIG. 7d). Mass spectrometry analysis of skin biopsies retrieved at day 14 from animals treated with 10 μM and 100 μM galunisertib (N=3 per condition) validates the presence of the compound at the concentrations of 1.6±0.7 μM and 9.6±0.6 μM, respectively (FIG. 7e).

The effect of the pharmacological treatments is seen also in the HF growth cycle. Harvested dorsal skin for comparative histological analysis at days 0, 5, 7, 14, 16 and 18, through hematoxylin-eosin staining of transversal HF sections, reveals a significant increase in the number of HF in the galunisertib- and minoxidil-treated groups, from day 7 to day 14, in comparison to the control vehicle treatment (FIG. 8a,b). When specifically quantifying the number of HFs within the subcutaneous layer (anagenic HFs), an increased number of HFs is observed already at day 5 (FIG. 8c). Surprisingly, the increased number of anagenic HFs perdures until day 14 in the galunisertib but not in the minoxidil treatment, although at day 16 the anagenic HFs start decreasing in all treatment conditions, disappearing at day 18 (telogen) (FIG. 8c).

Overall, the data suggest that similarly to minoxidil, but more efficiently, galunisertib improves anagenic hair growth. An increased number of anagenic HFs is detected from day 5 to day 14 in galunisertib, which is in agreement with the trichoscopy analysis. Still, catagen onset appears not to be delayed as it followed similar timing in all conditions.

The present invention further discloses the effect of galunisertib on progenitor cell activation in the hair germ compartment. Performing immunohistochemistry analysis of the Ki67 proliferation marker at days 5, 7 and 14 post-treatment (FIG. 8d) demonstrates that both galunisertib and minoxidil treatments induce an increased number of Ki67-positive cells in the hair germ in comparison to the vehicle treatment (FIG. 8e), which is indicative of progenitor cell activation. Surprisingly, galunisertib reveals more effective than minoxidil at day 7.

Altogether, the present invention discloses the beneficial effect of galunisertib topical treatment for use on hair growth enhancement treatment of AGA, alopecia and hair loss, based on a mode of action in which Galunisertib inhibits TGFβ signaling in HFOCs (with decreased levels of the phosphorylated SMAD2 canonical target) and counteracts TGFβ2 accrual in the epithelial compartment, which translates into improved cell proliferation required to sustain anagen (while the apoptosis marker cleaved Caspase 3 in the DP remains unchanged). Thus, rather than inhibiting DP cell apoptosis, Galunisertib appears to inhibit TGFβ2 gene expression in the DP and its detrimental paracrine signaling in the adjacent epithelial compartment. Notably, Galunisertib is able to rescue the levels of the trichogenic markers, transcription factor LEF1 and β-CATENIN, which validates the increased proliferation of follicular keratinocytes, thereby attesting Galunisertib effect in delaying of catagen onset.

Given that TGFβ is a multifunctional pathway involved in the hair follicle cycle, skin healing process and regeneration, the present invention refers to Galunisertib for use in the treatment of AGA as well as in scarless hair restoration surgery.

Other embodiments of the present invention comprise any chemical derivative of Galunisertib.

In another embodiment the present invention refers to a pharmaceutical composition for use in the treatment of androgenic alopecia comprising Galunisertib and other compounds selected from the group consisting of an excipient, a diluent, a stabilizing agent, a carrier, a surfactant, an antioxidant and a buffering agent, or combinations thereof.

In another embodiment the present invention's pharmaceutical composition is characterized in that it is suitable for one or more routes of administration selected from the group consisting of topical, dermal, transdermal and subcutaneous.

### BRIEF DESCRIPTION OF THE FIGURES

FIG. 1. Galunisertib blocks TGFβ activation in human hair follicle organ cultures (HFOCs). a) Schematic representation of DHT and galunisertib treatments in human HFOCs. b) RT-qPCR analysis of TGFβ transcripts in dermal papilla isolated from HFOCs treated with DHT, galunisertib or DHT+galunisertib, for 48 h. Values are mean±SD of samples from 3 independent donors. c) Western blot and quantitative analysis of SMAD2/3 and phospho-SMAD2 protein levels as readout for canonical TGFβ signaling activation in HFOCs treated with DHT or DHT+galunisertib, for 72 h. GAPDH was used as loading control and for normalization. Ns not significant, *p≤0.05 by unpaired t-test (b) and paired t-test (c) statistical tests.

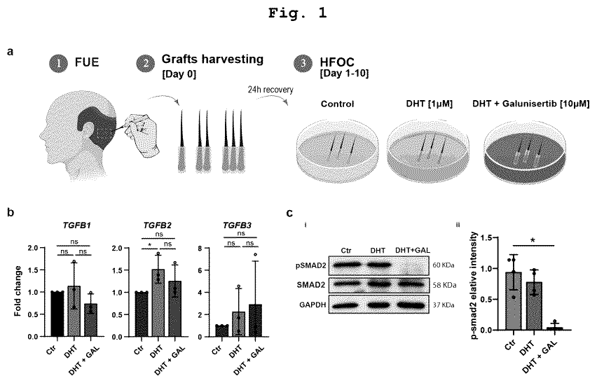

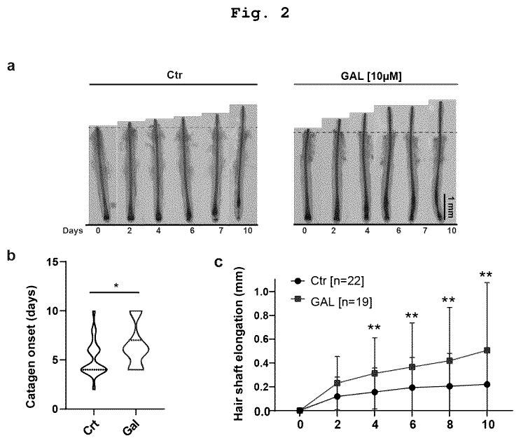

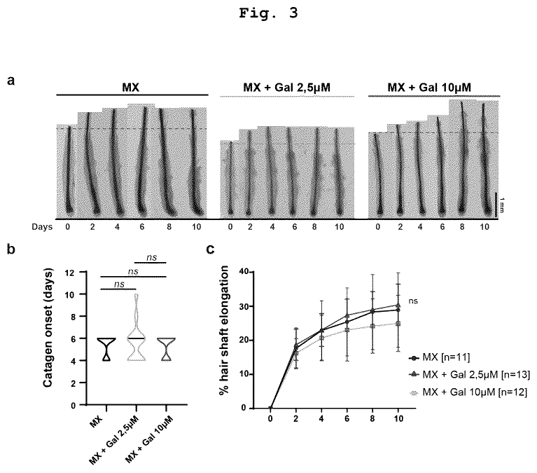

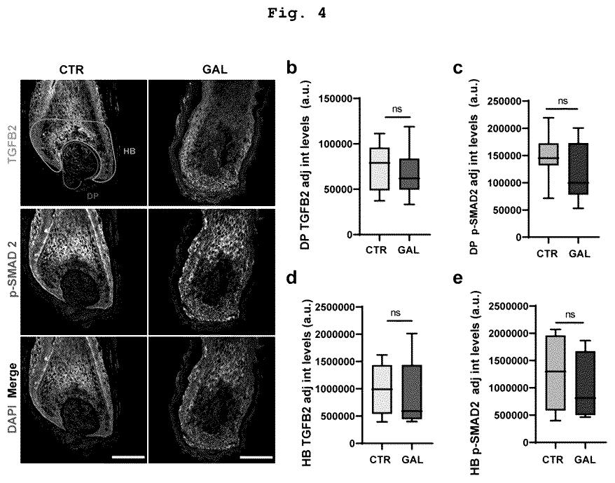

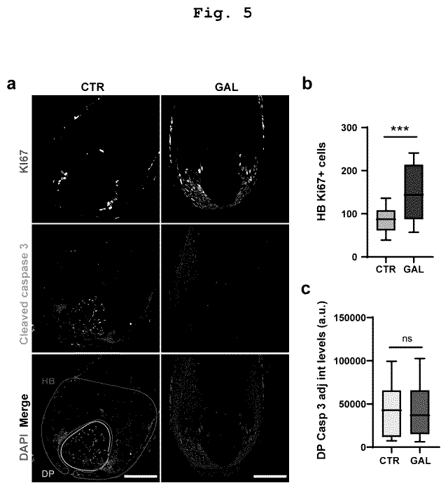

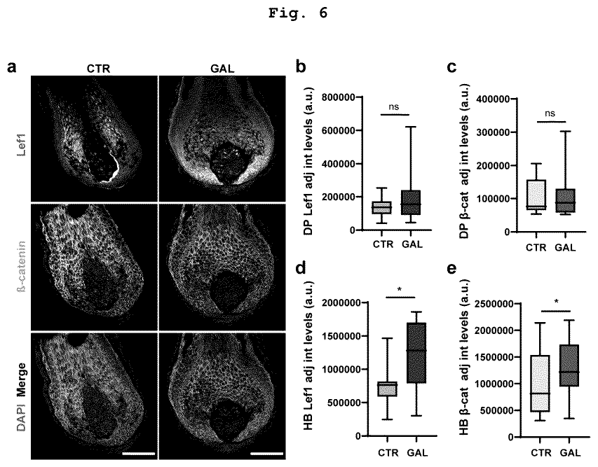

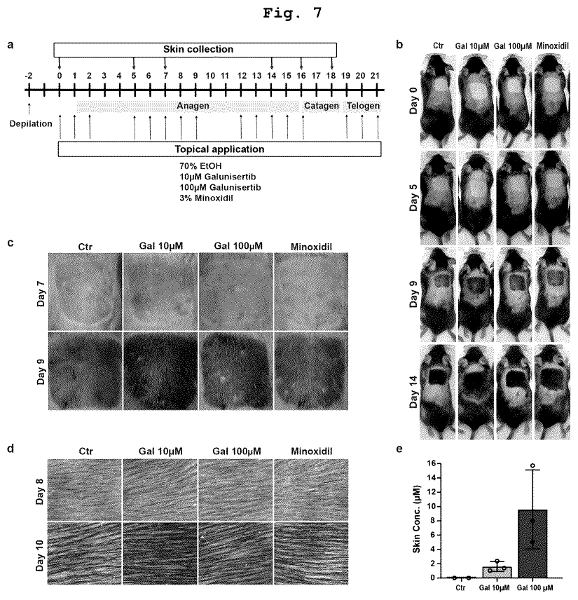

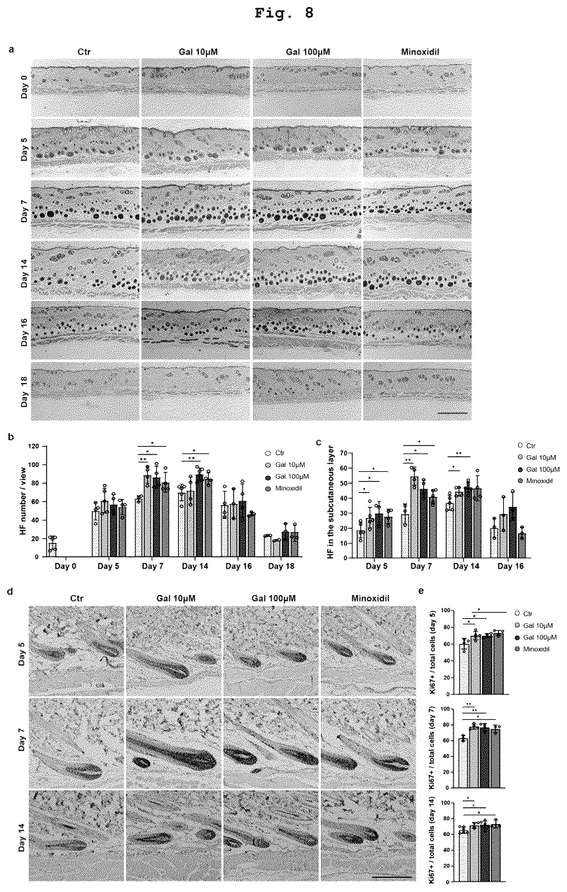

For RNA isolation and Quantitative Real-Time PCR (qPCR), Qiagen RNeasy kit (Qiagen, Germany) is used to isolate RNA. Concentration and purity are ascertained in a NanoDrop spectrophotometer. 600 ng of RNA are reverse transcribed into complementary DNA with iScript™ cDNA Synthesis Kit. Quantitative real-time polymerase chain reactions (qPCRs) are performed in a CFX384 Touch™ Real-Time PCR Detection System (Bio-Rad Laboratories). 10 μl reaction mixes are prepared consisting of iTaq™ Universal SYBR® Green Supermix (Bio-Rad Laboratories), forward and reverse primers (at 125 nM each) and cDNA (30 ng). The amplification program used is: 95° C. for 3 min, followed by 39 cycles of 95° C. for 10 s, 60° C. for 35 s, and melt curve from 55° C. to 95° C. with 0.5° C. increment for 10 s. All primers are designed to span at least one exon-intron junction. Transcript levels are quantified by the 2-ΔΔCt method. ΔCt values are determined by subtracting the geometrical mean of the Ct values for GAPDH and UBQ from the mean target gene Ct value.

For western blot, hair bulbs are dissected from HFOCs treated with DMSO, DHT and Galunisertib for 72 h. Upon rinse in PBS, hair bulbs are lysed in Laemmli buffer (without bromophenol blue) with Halt Protease Inhibitor Cocktail and Halt Phosphatase Inhibitor Cocktail, and lysates are sonicated. Lysates from 10 hair bulbs are used for each condition tested. Total extracts are then loaded in SDS-polyacrylamide gels and transferred onto nitrocellulose membranes for western blot analysis. Membranes are blocked during 1 h with TBS+5% BSA. Primary and secondary antibodies are diluted in TBS+2% BSA accordingly to Supplementary Table 1. Signal is detected using Clarity Western ECL Substrate reagent. A GS-800 calibrated densitometer with Quantity One 1-D Analysis Software 4.6 (Bio-Rad Laboratories) is used for quantitative analysis of protein levels.

FIG. 2. Galunisertib delays catagen entry in HFOCs. a) Hair growth ex vivo (HFOCs) under different treatment conditions for 10 days. Representative HF photos for each condition over time are shown in the panels. DP detachment was used as readout for catagen onset. Scale bar, 1 mm. b) Catagen onset timing in HFOCs under different treatment conditions as indicated. c) Percentage of hair shaft elongation in comparison to day 0. In b) and c), values are mean±SD of HFOCs established from n=5 independent donors (n>20 HFs per condition). ns not significant, *p≤0.05, **p≤0.01 by Mann-Whitney (b,c).

Hair grafts are obtained from male patients aged between 18-55 years with informed consent. A total of 10-20 micrografts are harvested from the occipital scalp of patients diagnosed with androgenic alopecia and undergoing autologous hair transplantation by the routine FUE technique. All micrografts used are intact, with visible amount of fat and connective tissue. In addition, only anagen hair follicles are used.

Ex vivo human scalp hair follicle organ culture, hair follicle units (HFs) from human adult scalp tissue are cultured in Williams E medium supplemented with 10 ng/ml hydrocortisone, 10 g/ml insulin, 2 mM 1-glutamine and 1× antibiotic-antimycotic. HFs are maintained at 37° C. in 5% C02 atmosphere. Minoxidil (M4145, Merck Life Science) is used at 1 μM final concentration, Galunisertib (S2230, obtained from Selleck Chemicals CO) at 10 μM optimal concentration, and DHT (D-073, obtained from Merck Life Science) at 1 μM. Culture medium is replaced every 2 days, and images of individual follicles are collected under a Leica M80 stereomicroscope (Leica Microsystems). HFs elongation is measured from images acquired over 10 days using the opensource ImageJ software. Hair growth percentages is normalized to day 0.

FIG. 3. Evaluation of combination treatment with Galunisertib and Minoxidil in HFOCs. a) HFOCs treatment with Minoxidil and Galunisertib for 10 days to evaluate drug synergic effect. b) Catagen onset timing in HFOCs treated as indicated. c) Percentage of hair shaft elongation in comparison to day 0. In b) and c), values are mean±SD of HFOCs established from n=2 independent donors (n>10 HFs per condition). ns not significant, *p≤0.05 by Mann-Whitney statistical test.

FIG. 4. Galunisertib inhibits TGFβ2 paracrine signaling in the bulb epithelial compartment. a) Representative Immunohistochemistry (IHC) images of tissue sections of HFOCs untreated and treated with Galunisertib as indicated and stained for TGFβ2 and p-SMAD2. In the upper left frame is exemplified how the dermal papilla (DP) and adjacent bulb epithelial compartment (HB) regions were delineated for quantitative analyses shown in b-e. Nuclei were counterstained with DAPI. Scale bar, 100 μm. b-e) Adjusted intensity levels of TGFβ2 and p-SMAD2 per DP and HB areas as measured in tissue sections from untreated and Galunisertib-treated HFOCs. Values are mean±SD of HFOCs established from n=4 donors (n>9 HFs per condition). ns p≥0.05, *p≤0.05 by unpaired t-test.

FIG. 5. Galunisertib improves cell proliferation and the WNT signaling in the bulb epithelial compartment. a) Representative IHC images of tissue sections of HFOCs untreated and treated with Galunisertib and stained for KI67 and cleaved caspase 3. Nuclei were counterstained with DAPI. Scale bar, 100 μm. b,c) Adjusted intensity levels of KI67 and cleaved caspase 3 per DP and HB (bulb epithelium) areas, respectively, as measured in tissue sections from HFOCs with and without Galunisertib. All values are mean±SD of HFOCs established from n=8 donors (n>16 HFs per condition). ns p≥0.05, *p≤0.05 by unpaired t-test.

FIG. 6. Galunisertib improves the WNT signaling in the bulb epithelial compartment. a) Representative IHC images of tissue sections of HFOCs with and without Galunisertib and stained for LEF1 (red) and β-catenin (green). Nuclei were counterstained with DAPI (blue). Scale bar, 100 μm. b,e) Adjusted intensity levels of LEF1 and β-catenin per DP and HB (bulb epithelium) areas, as measured in tissue sections from HFOCs with and without Galunisertib. All values are mean±SD of HFOCs established from n=4 donors (n>8 HFs per condition). ns p≥0.05, *p≤0.05 by unpaired t-test.

For immunohistochemistry, 2-day HFOCs are used for immunohistochemistry analysis. Briefly, HFs were embedded on a drop of OCT and stored at −80° C. 8 μm cryosections are air-dried and fixed in 4% PFA (for TGFβ2, cleavage Caspase 3, p-SMAD2 and KI67 immunostainings) or cold acetone (for LEF1 and β-catenin immunostaining). Following 2×5 min rinse with PBS, microsections are permeabilized 2× for 10 min with PBS+0.3% Triton X 100 (PBS-T) and then blocked with PBS+10% fetal bovine serum (FBS) for 1 hour at room temperature. Antibodies are diluted in PBS-T+1% FBS and incubation done overnight at 4° C. Upon 2×5 min rinse with PBS-T, samples are incubated with the secondary antibodies, goat anti-human Alexa-488 (1:1000; Life Technologies) and goat anti-human Alexa-568 (1:1000; Life technologies). Nuclei are counterstained with DAPI (Sigma-Aldrich), and slides are then mounted with 90% glycerol, 0.5% N-propyl-gallate, 20 nM Tris, pH 8 solution. All images are acquired under a Laser Scanning Confocal Microscope (Leica TCS SP5 II, Leica Microsystems) operated by the Leica Confocal Software—LAS, using 40×/1.1 water immersion objective and an acquisition speed of 400 Hz.

FIG. 7. Galunisertib induces hair growth in C57BL/6J mice. a) Mouse treatment scheme. 7-week-old C57BL/6J mice were depilated (day −2) and 2 days later (day 0) started being treated with vehicle (70% ethanol), galunisertib (10 μM and 100 μM) or 3% minoxidil for 21 non-continuous days. b) Photographs of C57BL/6J mice treated with vehicle (control), 10 μM and 100 μM galunisertib or 3% minoxidil at days 0, 5, 9 and 14. c) Magnified photographs of the depilation site of C57BL/6J mice treated with vehicle (Ctr), galunisertib (10 and 100 μM) or minoxidil at days 7 and 9. d) Trichoscopy images of depilated regions at days 8 and 10. N≥3 animals were used per timepoint per condition for image and skin collection; e) Mass spectrometry analysis of skin biopsies retrieved at day 14 from animals treated with 10 μM and 100 μM galunisertib. Values are mean±SD of n=3 animals per condition.

Mice are six-week-old male C57BL/6J mice purchased from the Charles River Laboratories. The animals are housed and allowed to adapt to the laboratory environment for one week before experiments start. Animals are maintained under a 12 h light/dark cycle and fed with regular rodent's chow and tap water ad libitum, at the i3S animal facility.

For depilation and treatments in order to synchronize hair growth cycle in mice, anagen is induced by depilation. Seven-week-old male C57BL/6J mice are anesthetized using 3% isoflurane and their back is shaved using a hair shaver. Hair is then further depilated using cold wax bands (Veet Minima). After the depilation, the excess of wax is removed with mineral oil, and a skin protector spray, Douxo Calm Sérum, is applied, followed by Cavilon, a barrier film spray. To allow for skin to fully recover, topical treatments started two days later. During the procedures mice are under volatile anesthesia (3% isoflurane) and are kept warmed. Mice are photographed with a camara or a trichoscope (Dino-Lite), before topical application of galunisertib (10 μM and 100 μM), minoxidil (3%) or vehicle (70% ethanol). Topical applications are performed using a syringe in order to cover the depilated area only. Hair growth is followed for 21 days, although telogen is apparent at day 18 post-treatment.

For mass spectrometry of skin biopsies, dorsal skin of mice under 10 μM and 100 μM galunisertib topical treatment for 14 days is collected after anesthesia (Ketamine 150 mg/kg+Medetomidine 2 mg/kg) and perfusion with PBS. Skin samples are snap frozen and stored at −80° C. until being sent for mass spec analysis by Cyprotex. Skin samples from N=3 animals per condition are homogenized 1:4 in mouse plasma, then quantified against a mouse plasma calibration curve. Analytical runs meeting calibration standard and QC acceptance criteria.

FIG. 8. Galunisertib increases anagenic hair follicle growth. a) Hematoxilin-eosin staining of transversal paraffin sections of the dorsal skin of mice treated with vehicle (control), galunisertib (10 μM and 100 μM) or minoxidil at days 0, 5, 7, 14, 16 and 18 of treatment. Scale bar, 500 μm. b) Number of hair follicles per field view and c) number of hair follicles per field view in the subcutaneous layer at different timepoints along the experiment. d) Ki67 immunostaining of longitudinal paraffin sections of the dorsal skin of mice treated with vehicle (control), galunisertib (10 μM and 100 μM) or minoxidil at days 5, 7 and 14. Scale bar, 200 μm. e) Number of Ki67-positive cells in the hair bulb at days 5, 7 and 14. N≥3 animals per timepoint per condition were analyzed, and N≥4 field views were quantified per animal. Values are mean±SD. *p<0.05; **p<0.01 by Student's t-test.

For histology and immunohistochemistry, dorsal skin from the depilated region is collected from mice at different timepoints across the experiment (days 0, 5, 7, 14 and 18) and fixed in 10% buffered formalin, imbedded in paraffin and sectioned along the transversal and longitudinal plan of the hair follicles (3 μm sections). Standard protocol is used for hematoxylin and eosin staining. For immunohistochemistry, slides are deparaffinized, then antigen retrieval is performed using HistoVT One (#06380-05, Nacalai Tesque Inc.) for 40 min in a steamer, following 20 min of cooling at room temperature (RT). After washing with TBS-0.01% Tween (TBS-T), peroxidase is blocked using Bloxall (#SP-6000, Vector Laboratories Inc.) for 10 min. Slides are then washed with TBS-T, and blocked using 2.5% Normal Horse Serum (#MP7500, Impress universal kit, Vector Laboratories Inc.) for 20 min. Following this, slides are incubated with rabbit anti-Ki67 antibody (D3B5) in 0.25% Normal Horse Serum overnight at 4° C. Next day, after thoroughly washing with TBS-T, the slides are incubated with a secondary anti-mouse/anti-rabbit antibody (#MP7500, Impress universal kit, Vector Laboratories Inc.) for 45 min RT. Impact DAB (#SK-4105, Vector Laboratories Inc.) are used to develop the slides, followed by dehydration and mounting with Entellan (#1.07961.0100, Millipore) following standard procedures.

In statistical analysis, sample size and statistical tests used in each experiment are indicated. Statistical analyses are performed with GraphPad Prism version 7. Data is tested for parametric or non-parametric distribution using D'Agostino-Pearson omnibus normality test. Two-tailed Mann-Whitney or t-test is used to determine statistical differences between groups ****p<0.0001, ***p<0.001, **p<0.01, and *p<0.05. All values are mean±SD

### REFERENCES

Adil, A., & Godwin, M. (2017). The effectiveness of treatments for androgenetic alopecia: A systematic review and meta-analysis. Journal of the American Academy of Dermatology, 77(1), 136-141.e135. doi:10.1016/j.jaad.2017.02.054
Cotsarelis, G. (2006). Epithelial stem cells: a folliculocentric view. J Invest Dermatol, 126(7), 1459-1468. doi:10.1038/sj.jid.5700376
Enshell-Seijffers, D., Lindon, C., Kashiwagi, M., & Morgan, B. A. (2010). beta-catenin activity in the dermal papilla regulates morphogenesis and regeneration of hair. Dev Cell, 18(4), 633-642. doi:10.1016/j.devcel.2010.01.016
Madaan, A., Verma, R., Singh, A. T., & Jaggi, M. (2018). Review of Hair Follicle Dermal Papilla cells as in vitro screening model for hair growth. International Journal of Cosmetic Science, 40(5), 429-450. doi:doi:10.1111/ics.12489
Martinez-Jacobo, L., Villarreal-Villarreal, C. D., Ortiz-López, R., Ocampo-Candiani, J., & Rojas-Martinez, A. (2018). Genetic and molecular aspects of androgenetic alopecia. Indian J Dermatol Venereol Leprol, 84(3), 263-268. doi:10.4103/ijdvl.IJDVL_262_17
Oh, J. W., Kloepper, J., Langan, E. A., Kim, Y., Yeo, J., Kim, M. J., Plikus, M. V. (2016). A Guide to Studying Human Hair Follicle Cycling In Vivo. J Invest Dermatol, 136(1), 34-44. doi:10.1038/jid.2015.354
Otberg, N., Finner, A. M., & Shapiro, J. (2007). Androgenetic alopecia. Endocrinol Metab Clin North Am, 36(2), 379-398. doi:10.1016/j.ecl.2007.03.004
Pantelireis, N., & Higgins, C. A. (2018). A bald statement—Current approaches to manipulate miniaturisation focus only on promoting hair growth. Experimental Dermatology, 27(9), 959-965. doi:https://doi.org/10.1111/exd.13690
Talavera-Adame, D., Newman, D., & Newman, N. (2017). Conventional and novel stem cell based therapies for androgenic alopecia. Stem Cells Cloning, 10, 11-19. doi:10.2147/sccaa.S138150
Vander Ark, A., Cao, J., & Li, X. (2018). TGF-β receptors: In and beyond TGF-β signaling. Cellular Signalling, 52, 112-120. doi:https://doi.org/10.1016/j.cellsig.2018.09.002
Winiarska, A., Mandt, N., Kamp, H., Hossini, A., Seltmann, H., Zouboulis, C. C., & Blume-Peytavi, U. (2006a). Effect of 5alpha-dihydrotestosterone and testosterone on apoptosis in human dermal papilla cells. Skin Pharmacol Physiol, 19(6), 311-321. doi:10.1159/000095251
Winiarska, A., Mandt, N., Kamp, H., Hossini, A., Seltmann, H., Zouboulis, C. C., & Blume-Peytavi, U. (2006b). Effect of 5α-Dihydrotestosterone and Testosterone on Apoptosis in Human Dermal Papilla Cells. Skin Pharmacology and Physiology, 19(6), 311-321. doi:10.1159/000095251
Lisbon, 19^{th} June 2023

1. A method for treatment of diseases selected from the list consisting of androgenic alopecia and any other types of hair loss comprising administering Galunisertinib.

2. The method according to claim 1, wherein said Galunisertinib is chemical derivative comprising formula:

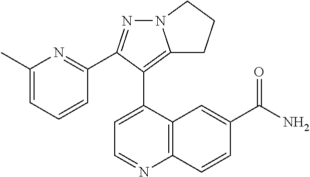 [SMILES: Cc1cccc(-c2nn3c(c2-c2ccnc4ccc(C(N)=O)cc24)CCC3)n1]

3. A pharmaceutical composition comprising galunisertinib and compounds selected from the group consisting of an excipient, a diluent, a stabilizing agent, a carrier, a surfactant, an antioxidant and a buffering agent, and combinations thereof.

4. The method according claim 2 wherein the Galunisertinib is administered topically, dermally, transdermally or subcutaneously.
